(19) United States
(12) Patent Application Publication
(10) Pub. No.: US 2025/0127951 A1
(43) Pub. Date: Apr. 24, 2025

(54) SILICA GEL FOAM DRESSING

(71) Applicant: Dukal, LLC, Ronkonkoma, NY (US)

(72) Inventor: La YANG, Dunedin, FL (US)

(21) Appl. No.: 18/726,014

(22) PCT Filed: Jan. 4, 2023
(86) PCT No.: PCT/US2023/010117

§ 371 Date: Jul. 1, 2024

(30) Foreign Application Priority Data
Jan. 4, 2022 (CN) 202220011172.9
Jan. 4, 2022 (CN) 202220011208.3

(51) Int. Cl. A61L 15/18; A61L 15/22; A61L 15/42

(52) U.S. Cl. CPC A61L 15/18; A61L 15/225; A61L 15/425

(57) ABSTRACT

A silica gel foam dressing includes a gluing carrier layer, a liquid absorption layer and a wound contact layer, wherein the liquid absorption layer is arranged between the gluing carrier layer and the wound contact layer, the wound contact layer is provided with a porous silica gel layer, and breathing pores. The liquid absorption layer may include a polyurethane foam layer and a sodium polyacrylate layer. The breathing pores may be of different and are formed in the wound contact layer to allow for absorbing wound exudates and improved breathability with large breathing pores provided over the wound and small breathing pores provided on the edges to limit warping and improve user comfort.

### CROSS-REFERENCE TO RELATED APPLICATIONS

The present application claims benefit of and priority to Chinese Patent Application No. 202220011208.3 filed Jan. 4, 2022 and Chinese Patent Application No. 202220011172.9 filed Jan. 4, 2022, the entire content of each of which is hereby incorporated by reference herein.

### BACKGROUND

### Field of the Disclosure

The present invention relates to medical dressings, and particularly relates to a silica gel foam dressing.

### Related Art

Medical dressings are used for treating common wounds, burns, bedsores, ulcers and the like. When in use, wound exudates are absorbed by wound contact layers of the dressings. Traditional dressing gauzes enable wound exudates to form dry scabs, moisture loss is remarkable, and the wound surfaces cannot be kept in a moist environment. Meanwhile, the traditional dressing gauzes are often bonded to the wound exudates, it is easily to cause mechanical loss when tearing off the traditional dressing gauzes, which is not conducive to the natural recovery of the wound, and a user will feel unbearable pain. In recent decades, with the proposing of the theory of moist healing and the development of biological materials, novel medical dressings have been emerging. In modern novel wound dressings, wound contact layers may contain hydrogel or silica gel. These dressings may provide a moist healing environment, protect the wound surface, relieve the pain of the wound and absorb a large amount of exudates.

However, if a silica gel layer is used as the wound contact layer, it is needed to form breathing pores in the silica gel layer due to its water tightness. The breathing pores of the same size are formed in the contact layers in the most existing processing technologies. However, the use of breathing pores reduces application performance of the contact layer is reduced, the dressing is easily subjected to edge warping in the using process, bonding is difficult, and comfortable protection is difficult to achieve.

It would be beneficial to provide a silica gel foam dressing that avoids the above and other problems.

### SUMMARY

It is an object of the present disclosure to provide a silica foam dressing that absorbs wound exudate and has a high application performance.

A silica gel foam dressing in accordance with an embodiment of the present disclosure includes: a gluing carrier layer; a liquid absorption layer; and a wound contact layer, wherein the liquid absorption layer is arranged between the gluing carrier layer and the wound contact layer, and the wound contact layer is provided with a porous silica gel layer.

In embodiments, the silica gel foam dressing includes a plurality of breathing pores with different apertures formed in the wound contact layer.

In embodiments, an aperture of the breathing pores ranges from 0.5 mm to 5.0 mm.

In embodiments, the plurality of breathing pores include a first group of large aperture breathing pores and a second group of small aperture breathing pores, wherein a size of an aperture of the large aperture breathing pores is larger than that of the small aperture breathing pores.

In embodiments, the large aperture breathing pores are formed in a wound contact area of the wound contact layer, and the small aperture breathing pores are formed in an application edge area of the wound contact layer.

In embodiments, the liquid absorption layer includes a first liquid absorption layer, a polyurethane foam layer and a sodium polyacrylate layer which are sequentially arranged in a direction from far to near from the wound contact layer.

In embodiments, the liquid absorption layer comprises a sodium polyacrylate layer and a polyurethane foam layer.

In embodiments, the liquid absorption layer is one of a sodium polyacrylate layer and a polyurethane foam layer.

In embodiments, the first liquid absorption layer is at least one of a polyurethane foam layer, a knitted fabric layer and a non-woven fabric layer.

In embodiments, a transverse size of the wound contact layer is larger than that of the liquid absorption layer, wherein respective transverse sizes of all layers in the liquid absorption layer are gradually increased in a direction from far to near from the wound contact layer, and the respective transverse sizes of whole edges of the wound contact layer and the liquid absorption layer are regularly increased gradually in a direction from far to near from the wound contact layer.

In embodiments, the transverse size of the wound contact layer is larger than that of the liquid absorption layer, and transverse sizes of the whole edges of the wound contact layer and the liquid absorption layer are regularly increased gradually in a direction from far to near from the wound contact layer.

In embodiments, the size difference between all the layers in the liquid absorption layer is 0-30 mm.

In embodiments, the gluing carrier layer includes a polyurethane film carrier layer.

In embodiments, the gluing carrier layer includes a pressure-sensitive adhesive layer, and the pressure-sensitive adhesive layer is arranged between the polyurethane film carrier layer and the liquid absorption layer.

In embodiments, a bonding layer is arranged between the first liquid absorption layer and the sodium polyacrylate layer.

In embodiments, the wound contact layer includes a wound contact area and an application edge area.

In embodiments, the wound contact layer includes a porous pressure-sensitive adhesive layer and a porous polyurethane film layer, the porous pressure-sensitive adhesive layer is arranged on a face of the porous polyurethane film layer close to the liquid absorption layer, and the porous silica gel layer is arranged on the other face of the porous polyurethane film layer.

In embodiments, the plurality of breathing pores extend through the porous pressure-sensitive adhesive layer, the porous polyurethane film layer and the porous silica gel layer.

In embodiments, the wound contact layer includes a wound contact area and an application edge area.

In embodiments, the wound contact area corresponds to an area closest to the wound contact layer in all the layers in the liquid absorption layer.

In embodiments, the silica gel foam dressing includes a release film layer arranged on an outer side of the porous silica gel layer.

In embodiments, the pressure-sensitive adhesive layer includes an acrylic adhesive structure.

In embodiments, the acrylic adhesive structure is an acrylic adhesive.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates a top view of a silica gel foam dressing in accordance with an embodiment of the present disclosure;

FIG. 2a illustrates a cross-sectional view of a silica gel foam dressing in accordance with an embodiment of the present disclosure;

FIG. 2b illustrates a cross-sectional view of a silica foam dressing in accordance with another embodiment of the present disclosure;

FIGS. 3a-3d illustrate cross-sectional views of a silica gel foam dressing in accordance with embodiments of the present disclosure;

FIG. 4a illustrates a top view of a silica gel foam dressing in accordance with an embodiment of the present disclosure; and

FIG. 4b illustrates a cross-sectional view of the silica gel foam dressing of FIG. 4a.

### DESCRIPTION OF REFERENCE NUMERALS

1—polyurethane film carrier layer; 2—pressure-sensitive adhesive layer; 3—first liquid absorption layer; 4—bonding layer; 5—sodium polyacrylate layer; 6—polyurethane foam layer; 7—porous pressure-sensitive adhesive layer; 8—porous polyurethane film layer; 9—porous silica gel layer; 10—release film layer; 11—large breathing pore; and 12—small breathing pore.

### DETAILED DESCRIPTION OF THE EXEMPLARY EMBODIMENTS

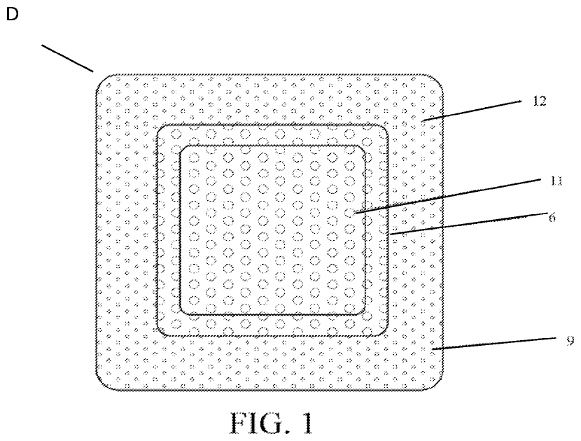

Specific implementations of a silica gel foam dressing D (see FIG. 1, for example) are further described below in conjunction with the accompanying drawings and embodiments. The following embodiments are only used to more clearly illustrate the present invention and are not intended to limit scope of protection provided by the claims.

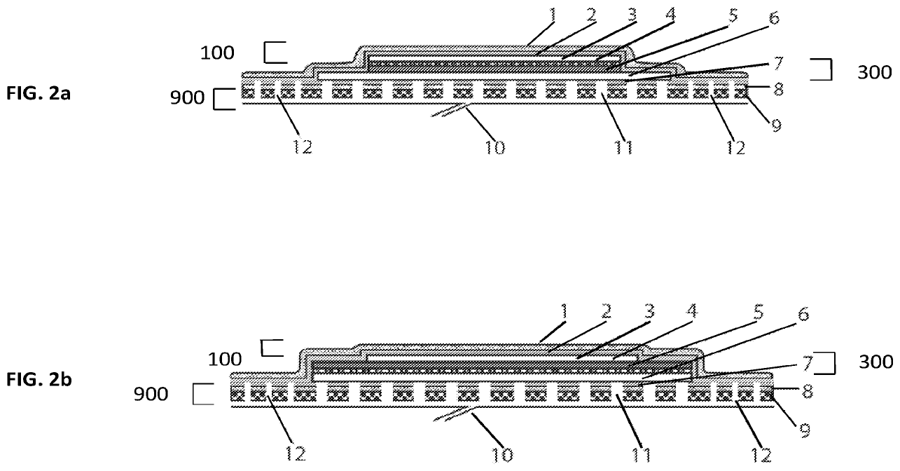

A silica gel foam dressing D, in embodiments, may include a gluing carrier layer 100, a liquid absorption layer 300 and a wound contact layer 900, wherein the liquid absorption layer is arranged between the gluing carrier layer and the wound contact layer as can generally be seen in FIGS. 2a-2b, 3a-3d and 4b, for example. In embodiments, the wound contact layer 900 may include a porous silica gel layer 9, as illustrated in FIGS. 2a-2b, 3a-3d and 4b, for example, and breathing pores 11, 12 (see FIGS. 1 and 2a-2b, for example) with different apertures formed therein.

With the adoption of the arrangement of the structure above, it can be guaranteed that the liquid absorption layer 300 has good liquid absorption capacity and breathability through the breathing pores 11, 12, and the application performance of the wound contact layer 900 using small aperture pores 12 is improved. In addition, in embodiments, the wound contact layer 900 bonds to the skin, and is mild and free of stimulation, and may be repeatedly bonded, and restrains scar spasticity and prevents hyperplastic scars.

In embodiments, the liquid absorption layer 300 may include multiple layers and may be embodied in different combinations to achieve the absorption capacity and provide for use with wounds of different types.

In embodiments, the liquid absorption layer 300 includes a first liquid absorption layer 3, a polyurethane foam layer 6 and a sodium polyacrylate layer 5 as illustrated in FIGS. 2a-2b, for example. In embodiments, arranging the polyurethane foam layer 6 and the sodium polyacrylate layer 5 provides absorption capacity for wound exudates that is ultrahigh. In embodiments, when the liquid absorption capacity is good, the wound is kept in a moist environment, and the wound rapidly heals.

In embodiments, the liquid absorption layer 300 includes the first liquid absorption layer 3, the sodium polyacrylate layer 5 and the polyurethane foam layer 6 which are arranged from top to bottom as shown in FIG. 2a. In embodiments, the first liquid absorption layer 3 is positioned above the sodium polyacrylate layer 5 and the sodium polyacrylate layer 5 is positioned above the polyurethane foam layer 6 and these three layers jointly form the liquid absorption layer 300.

In embodiments, the first liquid absorption layer 3 may be polyurethane foam, a knitted fabric or a non-woven fabric. In embodiments, the size of the polyurethane foam layer 6 close to the wound contact layer 900 is slightly larger than that of the first liquid absorption layer 3. In embodiments, the size difference between the polyurethane foam layer 6 and the first liquid absorption layer 3 in width ranges from 0 mm to 30 mm. In embodiments, the size of the sodium polyacrylate layer 5 may be basically the same as that of the first liquid absorption layer 3 as shown in FIG. 2a, for example. In embodiments, the size of the sodium polyacrylate layer 5 may be larger than that of the first liquid absorption layer 3 as shown in FIG. 2b, for example. In embodiments, under normal conditions, the size of the wound contact layer 900 is the largest so that the dressing D may be bonded to the skin, the width from the liquid absorption layer 300 to the wound contact layer is increased layer by layer, and finally a step shape is formed in a longitudinal direction. In embodiments, a high buffer between the carrier layer and a contact application layer, so that the edge of the dressing D is not prone to warping when the dressing is used, and the more fitting technical effect may be achieved.

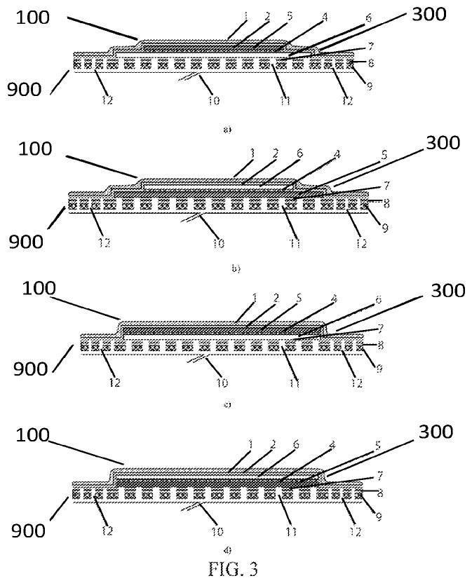

In embodiments, the liquid absorption layer 300 may include one polyurethane foam layer 6 and one sodium polyacrylate layer 5 and may not include the first liquid absorption layer 3 as can be seen in FIGS. 3a, 3b, 3c and 3d. In embodiments, arranging the sodium polyacrylate layer 5, the absorption capacity for wound exudates is ultrahigh, the liquid absorption capacity is good, and the wound is kept in a moist environment, and the wound can rapidly heal.

In embodiments, a size difference may exist between the polyurethane foam layer 6 and the sodium polyacrylate layer 5 as can be seen in FIGS. 3a and 3b. In embodiments, the same size may be kept between the polyurethane foam layer 6 and the sodium polyacrylate layer 5 as can be seen in FIGS. 3c and 3d.

In embodiments, the size difference between the polyurethane foam layer 6 and the sodium polyacrylate layer 5 width ranges from 0 mm to 30 mm.

In embodiments, a bonding layer 4 may be arranged between the polyurethane foam layer 6 and the sodium polyacrylate layer 5. In embodiments, the bonding layer 4 may not be used. In embodiments, the polyurethane foam layer 6 and the sodium polyacrylate layer 5 may be tightly attached together without the need for the bonding layer 4.

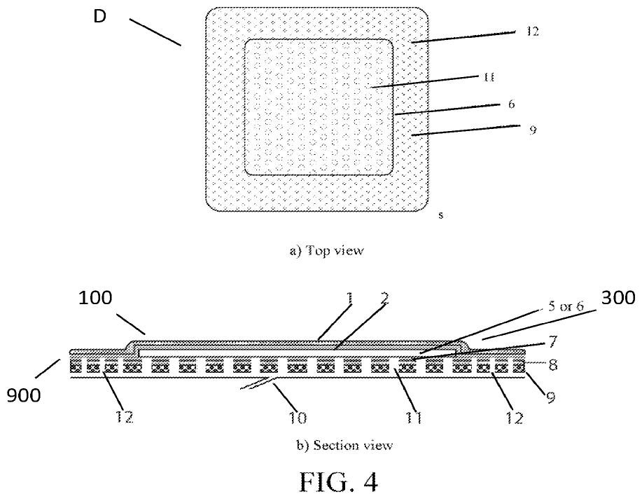

In embodiments, the liquid absorption layer 300 may include either one polyurethane foam layer 6 or one sodium polyacrylate layer 5 and may not include the first liquid absorption layer 3 as can be seen in FIGS. 4b, for example. In embodiments, the liquid absorption performance of the polyurethane foam layer 6 or the sodium polyacrylate layer 5 meets the absorption requirement of general wound exudate, and provides increased softness and comfort of the dressing D.

In embodiments, the gluing carrier layer 100 may include the polyurethane film carrier layer 1. In embodiments, polyurethane film (PU film) has very good elasticity, waterproofness and breathability and has antibacterial properties.

In embodiments, the gluing carrier layer 100 may include a pressure-sensitive adhesive layer 2 which may be arranged between the polyurethane film carrier layer 1 and the liquid absorption layer 300. In embodiments, the liquid absorption layer 300 may be bonded and fixed on the carrier layer 100 through the pressure-sensitive adhesive layer 2. In embodiments, the pressure-sensitive adhesive may be any one of an acrylic pressure-sensitive adhesive, a polyurethane pressure-sensitive adhesive or a medical polyisobutylene pressure-sensitive adhesive, to name a few.

Preferably, the pressure-sensitive adhesive layer 2 may use acrylic adhesive.

In embodiments, the bonding layer 4 may be arranged between the first liquid absorption layer 3 and the sodium polyacrylate layer 5. In embodiments, the bonding layer 4 may not be arranged between the first liquid absorption layer 3 and the sodium polyacrylate layer 5 and the first liquid absorption layer and the sodium polyacrylate layer may only need to be tightly attached together without the need for bonding layer 4.

In embodiments, the wound contact layer 900 may include a porous pressure-sensitive adhesive layer 7 and a porous polyurethane film layer 8, wherein the porous pressure-sensitive adhesive layer 7 is arranged on a face of the porous polyurethane film layer 8 close to the liquid absorption layer 300, and the porous silica gel layer 9 may be arranged on the other face of the porous polyurethane film layer 8 closer to the wound when the dressing D is in place. In embodiments, the liquid absorption layer 300 is arranged between the polyurethane film carrier layer 1 coated with the pressure-sensitive adhesive layer 2 and the porous polyurethane film layer 8 coated with the porous pressure-sensitive adhesive layer 7 on one face. In embodiments, the liquid absorption layer 300 and the wound contact layer 900 are bonded together through the porous pressure-sensitive adhesive layer 7, and the porous polyurethane film layer 8 is attached to a wound of a user through the porous silica gel layer 9, so that good comfort is achieved.

In embodiments, breathing pores 11, 12 may be formed in the porous pressure-sensitive adhesive layer 7, the porous polyurethane film layer 8 and the porous silica gel layer 9 and are in communication with each other such that the breathing pores extend through the porous silica gel layer, the porous pressure-sensitive adhesive layer and the porous polyurethane film layer. Therefore, in embodiments, the breathing pores 11, 12 extend from top to bottom through the whole wound contact layer 900 to provide ultrahigh breathability. In embodiments, the liquid absorption layer 300 fully absorbs exudative mucus of the wound through the breathing pores 11, 12 which are in communication with each other from top to bottom in the wound contact layer 900.

In embodiments, the breathing pores range from 0.5 mm to 5.0 mm. In embodiments, large breathing pores 11 and small breathing pores 12 may be used, wherein the large breathing pores 11 are formed in a wound contact area of the wound contact layer 900 and the small breathing pores 12 are formed in an application edge area of the wound contact layer 900 around a periphery of the wound contact area. In embodiments, the porous pressure-sensitive adhesive layer 7, the porous polyurethane film layer 8 and the porous silica gel layer 9 in the wound contact layer 900 are correspondingly provided with the large breathing pores 11 and the small breathing pores 12. Through the arrangement, the large breathing pores 11 in the wound contact area can ensure that the liquid absorption layer 300 quickly absorbs wound exudates, the small breathing pores 12 in the application edge area on an outer side of the wound contact area 900 prevents the edge of the dressing D from warping, and therefore the breathability and ability to properly apply and maintain the dressing in place over a variety of wounds are improved.

In embodiments, the aperture of the large breathing pores 11 ranges from 0.5 mm to 5.0 mm, and the aperture of the small breathing pores 12 ranges from 0.1 mm to 2.0 mm.

In embodiments, the large breathing pores 11 correspond to an area covered by the polyurethane foam layer 6. Therefore, the large breathing pores 11 are uniformly formed in the area corresponding to the polyurethane foam layer 6 and may be connected with a second polyurethane foam layer, and as a result, the liquid absorption capacity is guaranteed to the maximum extent, and the breathability is very high.

In embodiments, a release film 10 may be arranged on an outer side of the porous silica gel layer 9. In embodiments, the release film 10 may be attached to the silica gel layer 9 and protects the dressing from dust and other debris when the dressing is not in use. In embodiments, when the dressing D is used, the release film 10 is removed and the dressing covers and is bonded to the skin of the wound and around the wound.

In embodiments, the silica gel foam dressing D includes the polyurethane film carrier layer 1, the pressure-sensitive adhesive layer 2, the first liquid absorption layer 3, the bonding layer 4, the sodium polyacrylate layer 5, the polyurethane foam layer 6, the porous pressure-sensitive adhesive layer 7, the porous polyurethane film layer 8, the porous silica gel layer 9 and the release film layer 10 which are arranged from top to bottom. In embodiments, the silica gel foam dressing D has a high capacity to rapidly absorb exudate with high breathability and improved performance to provide a more user friendly performance. As noted above, however, the absorption layer 300 may include just the sodium polyacrylate layer 5 and the polyurethane foam layer 6 or either of the sodium polyacrylate layer 5 and the polyurethane foam layer 6. Further, as noted above, the bonding layer 4 may not be used.

The above description of the disclosed embodiments will enable those skilled in the art to realize or use this application. The general principles defined herein can be implemented in other embodiments without departing from the spirit or scope of this application. Therefore, this application will not be limited to the embodiments shown herein, but should conform to the widest scope consistent with the principles and novel features disclosed herein.

The above descriptions are merely specific examples of the present invention and experimental examples for proving the inventive concept of the invention application, and are not intended to limit the present invention. All transformations which can be directly derived from or associated with the contents disclosed in the present invention by a person of ordinary skill in the art shall be considered to be included in the protection scope of the present invention.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

What is claimed is:

1. A silica gel foam dressing, comprising:
a gluing carrier layer;
a liquid absorption layer; and
a wound contact layer,
wherein the liquid absorption layer is arranged between the gluing carrier layer and the wound contact layer, and
the wound contact layer is provided with a porous silica gel layer.

2. The silica gel foam dressing of claim 1 further comprising a plurality of breathing pores with different apertures formed in the wound contact layer.

3. The silica gel foam dressing of claim 2, wherein an aperture of the breathing pores ranges from 0.5 mm to 5.0 mm.

4. The silica gel foam dressing of claim 2, wherein the plurality of breathing pores include a first group of large aperture breathing pores and a second group of small aperture breathing pores, wherein a size of an aperture of the large aperture breathing pores is larger than that of the small aperture breathing pores.

5. The silica gel foam dressing of claim 4, wherein the large aperture breathing pores are formed in a wound contact area of the wound contact layer, and the small aperture breathing pores are formed in an application edge area of the wound contact layer.

6. The silica gel foam dressing of claim 1, wherein the liquid absorption layer comprises a first liquid absorption layer, a polyurethane foam layer and a sodium polyacrylate layer which are sequentially arranged in a direction from far to near from the wound contact layer.

7. The silica gel foam dressing of claim 1, wherein the liquid absorption layer comprises a sodium polyacrylate layer and a polyurethane foam layer.

8. The silica gel foam dressing of claim 1, wherein the liquid absorption layer is one of a sodium polyacrylate layer and a polyurethane foam layer.

9. The silica gel foam dressing of claim 6, wherein the first liquid absorption layer is at least one of a polyurethane foam layer, a knitted fabric layer and a non-woven fabric layer.

10. The silica gel foam dressing of claim 6, wherein a transverse size of the wound contact layer is larger than that of the liquid absorption layer,
wherein respective transverse sizes of all layers in the liquid absorption layer are gradually increased in a direction from far to near from the wound contact layer, and
the respective transverse sizes of whole edges of the wound contact layer and the liquid absorption layer are regularly increased gradually in a direction from far to near from the wound contact layer.

11. The silica gel foam dressing of claim 10, wherein the transverse size of the wound contact layer is larger than that of the liquid absorption layer, and transverse sizes of the whole edges of the wound contact layer and the liquid absorption layer are regularly increased gradually in a direction from far to near from the wound contact layer.

12. The silica gel foam dressing of claim 11, wherein the size difference between all the layers in the liquid absorption layer is 0-30 mm.

13. The silica gel foam dressing of claim 1, wherein the gluing carrier layer comprises a polyurethane film carrier layer.

14. The silica gel foam dressing of claim 1, wherein the gluing carrier layer further comprises a pressure-sensitive adhesive layer, and the pressure-sensitive adhesive layer is arranged between the polyurethane film carrier layer and the liquid absorption layer.

15. The silica gel foam dressing of claim 6, wherein a bonding layer is arranged between the first liquid absorption layer and the sodium polyacrylate layer.

16. The silica gel foam dressing of claim 1, wherein the wound contact layer comprises a wound contact area and an application edge area.

17. The silica gel foam dressing of claim 2, wherein the wound contact layer further comprises a porous pressure-sensitive adhesive layer and a porous polyurethane film layer, the porous pressure-sensitive adhesive layer is arranged on a face of the porous polyurethane film layer close to the liquid absorption layer, and the porous silica gel layer is arranged on the other face of the porous polyurethane film layer.

18. The silica gel foam dressing of claim 17, wherein the plurality of breathing pores extend through the porous pressure-sensitive adhesive layer, the porous polyurethane film layer and the porous silica gel layer.

19. The silica gel foam dressing of claim 6, wherein the wound contact layer comprises a wound contact area and an application edge area.

20. The silica gel foam dressing of claim 19, wherein the wound contact area corresponds to an area closest to the wound contact layer in all the layers in the liquid absorption layer.

21. The silica gel foam dressing of claim 1, wherein a release film layer is arranged on an outer side of the porous silica gel layer.

22. The silica gel foam dressing of claim 17, wherein the pressure-sensitive adhesive layer includes an acrylic adhesive structure.

23. The silica gel foam dressing of claim 22, wherein the acrylic adhesive structure is an acrylic adhesive.
